(19) United States
(12) Patent Application Publication
(10) Pub. No.: US 2014/0025126 A1
(43) Pub. Date: Jan. 23, 2014

(54) Fixed Rod Clamping Structure for a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/555,638

(22) Filed: Jul. 23, 2012

(51) Int. Cl. A61B 17/88

(52) U.S. Cl. 606/86 A

(57) ABSTRACT

A fixed rod clamping structure for a minimally invasive surgery contains: a handle; a joint module including a first sprocket and a second sprocket engaging with the first sprocket, with the first sprocket connecting with the handle; a curved bar coupling with the second sprocket of the joint module; a fitting post connecting with the curved bar and having an orifice and a through hole, with the orifice being perpendicular to and communicating with the through hole and having inner threads formed on one end thereof adjacent to the curved bar; and a positioning column including a rotatable head, a pin, and outer threads formed on one end thereof adjacent to the rotatable head. The outer threads are screwed with the inner threads of the orifice.

### BACKGROUND OF THE INVENTION

1. Field of the Invention

The present invention relates to a fixed rod clamping structure for a minimally invasive surgery which is capable of adjusting a desired angle between a handle and a curved bar by ways of a joint module to retain a rigid sleeve and a flexible shaft between at least bone screw precisely.

2. Description of the Prior Art

A conventional fixed rod clamping structure can only be operated at a fixed angle, so during inserting a fixed rod of the fixed rod clamping structure, the fixed rod interferes with a patient's back to stop the minimally invasive surgery. In addition, a size of a front end of the fixed rod clamping structure can not be decreased to cause a wound in a large size, thus the wound recover slowly.

The present invention has arisen to mitigate and/or obviate the afore-described disadvantages.

### SUMMARY OF THE INVENTION

The primary object of the present invention is to provide a fixed rod clamping structure for a minimally invasive surgery which is capable of adjusting a desired angle to prevent from an interference with a patent's back in a spine treatment and corrective rehabilitation.

Another object of the present invention is to provide a fixed rod clamping structure for a minimally invasive surgery which greatly decreases a size thereof.

A fixed rod clamping structure for a minimally invasive in accordance with a preferred embodiment of the present invention contains:

a handle;

a joint module including a first sprocket and a second sprocket engaging with the first sprocket, and the first sprocket connecting with the handle;

a curved bar coupling with the second sprocket of the joint module;

a fitting post connecting with the curved bar and having an orifice and a through hole, wherein the orifice is perpendicular to and communicates with the through hole and has inner threads formed on one end thereof adjacent to the curved bar;

a positioning column including a rotatable head, a pin, and outer threads formed on one end thereof adjacent to the rotatable head, wherein the outer threads are screwed with the inner threads of the orifice.

Thereby, the features and advantages of the fixed rod clamping structure for the minimally invasive:

1. A desired angle between the handle and the curved bar is adjusting by ways of the joint module without generating an interference.

2. A size of the fixed rod clamping structure is decreased to minimize a wound.

3. The rigid sleeve and the flexible shaft of the fixed rod clamping structure are retained securely between bodies of the at least one bone screw.

### BRIEF DESCRIPTION OF THE DRAWINGS

FIG. 1 is a perspective view showing the exploded components of a fixed rod clamping structure for a minimally invasive surgery according to a preferred embodiment of the present invention.

FIG. 2 is a cross sectional view showing the assembly of the fixed rod clamping structure for the minimally invasive surgery according to the preferred embodiment of the present invention.

FIG. 3 is another cross sectional view showing the assembly of the fixed rod clamping structure for the minimally invasive surgery according to the preferred embodiment of the present invention.

FIG. 4 is a cross sectional view showing the operation of the fixed rod clamping structure for the minimally invasive surgery according to the preferred embodiment of the present invention.

FIG. 4A is an amplified cross sectional view showing a part of the operation of the fixed rod clamping structure for the minimally invasive surgery according to the preferred embodiment of the present invention.

FIG. 5 is another cross sectional view showing the operation of the fixed rod clamping structure for the minimally invasive surgery according to the preferred embodiment of the present invention.

FIG. 6 is also another cross sectional view showing the operation of the fixed rod clamping structure for the minimally invasive surgery according to the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

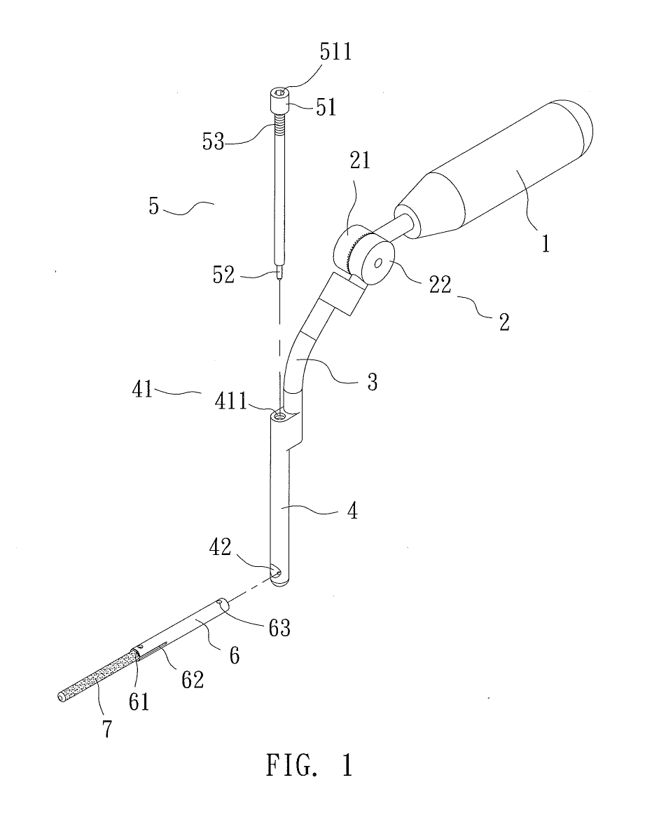

Referring to FIGS. 1-3, a fixed rod clamping structure for a minimally invasive surgery according to a preferred embodiment of the present invention comprises:

a handle 1;

a joint module 2 including a first sprocket 21 and a second sprocket 22 engaging with the first sprocket 21, and the first sprocket 21 connecting with the handle 1;

a curved bar 3 coupling with the second sprocket 22 of the joint module 2;

a fitting post 4 connecting with the curved bar 3 and having an orifice 41 and a through hole 42, wherein the orifice 41 is perpendicular to and communicates with the through hole 42 and has inner threads 411 formed on one end thereof adjacent to the curved bar 3;

a positioning column 5 including a rotatable head 51, a pin 52, and outer threads 53 formed on one end thereof adjacent to the rotatable head 51, wherein the outer threads 53 are screwed with the inner threads 411 of the orifice 41, and the rotatable head 51 of the positioning column 5 includes a hexagonal aperture 511.

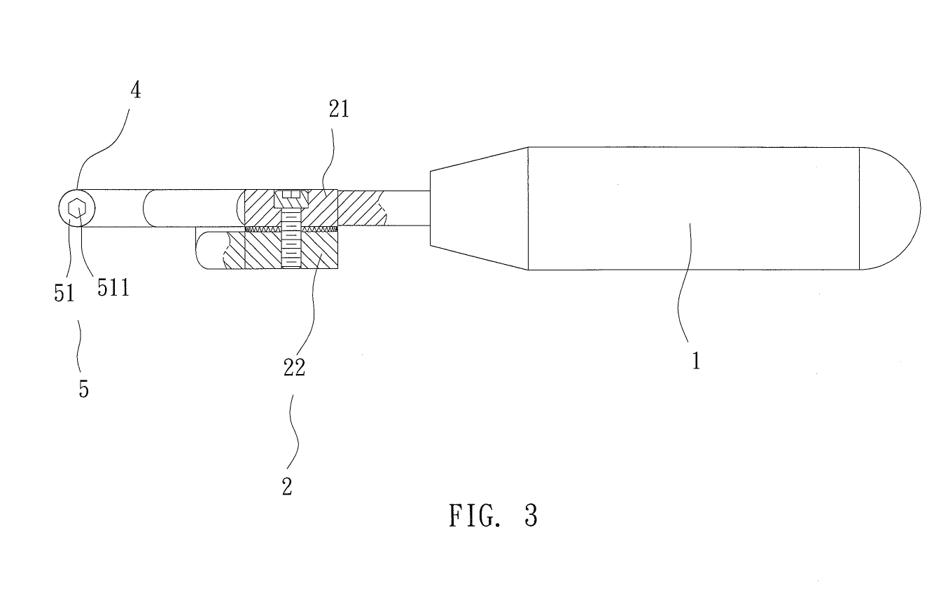

The handle 1 is axially connected with the curved bar 3 by ways of the first sprocket 21 and the second sprocket 22 of the joint module 2 as illustrated in FIG. 3.

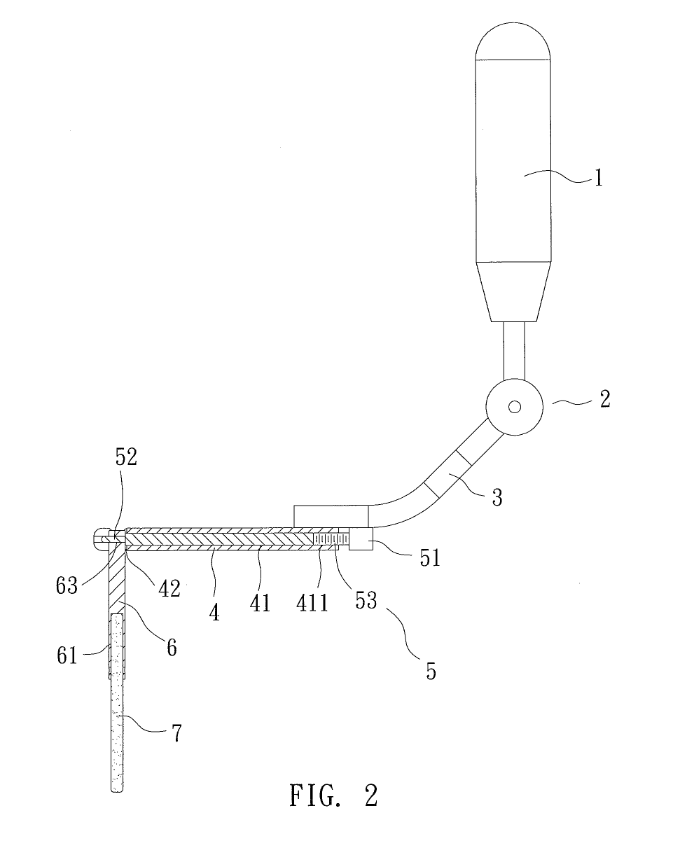

In assembly, a flexible shaft 7 is fitted in a receiving space 61 of a rigid sleeve 6, and the receiving space 61 has two slots 62 defined therein as shown in FIG. 1, the rigid sleeve 6 has an opening 63 defined on one end thereof which is inserted into the through hole 42 of the fitting post 4, and the positioning column 5 is inserted into the orifice 41 of the fitting post 4, and then a tool (such as a hexagon wrench) is fitted into the hexagonal aperture 511 of the rotatable head 51, thereafter the positioning column 5 is rotated so that the outer threads 53 of the positioning column 5 screw with the inner threads 411 of the orifice 41, hence the positioning column 5 is coupled with the fitting post 4 securely, and the pin 52 of the positioning column 5 is inserted into the opening 63 of the rigid sleeve 6 as shown in FIG. 2 so that the fixed rod clamping structure with the rigid sleeve 6 and the flexible shaft 7 is fixed stably.

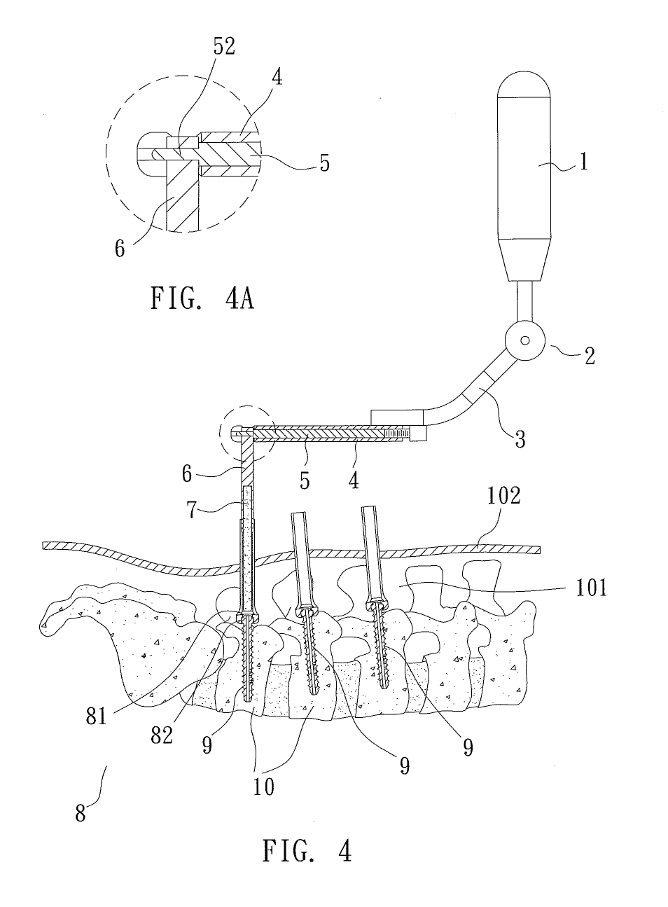

Referring further to FIGS. 4 and 4A, in operation, the joint module 2 is used to adjust a desired angle between the handle 1 and the curved bar 3, and the positioning column 5 is fitted into the fitting post 4 so that the pin 52 of the positioning column 5 connects with the opening 63 of the rigid sleeve 6, hence the fixed rod clamping structure with the rigid sleeve 6 and the flexible shaft 7 is fixed securely. Thereafter, the flexible shaft 7 is inserted into a skin 102 perpendicularly and further reaches a position where a holder 81 and an axial seat 82 of a body 8 of at last one bone screw are located via a minor wound and a muscle tissue 101, in the meantime, at least one screw section 9 on a front end of the body 8 of the at least one bone screw screws with at least one spine 10.

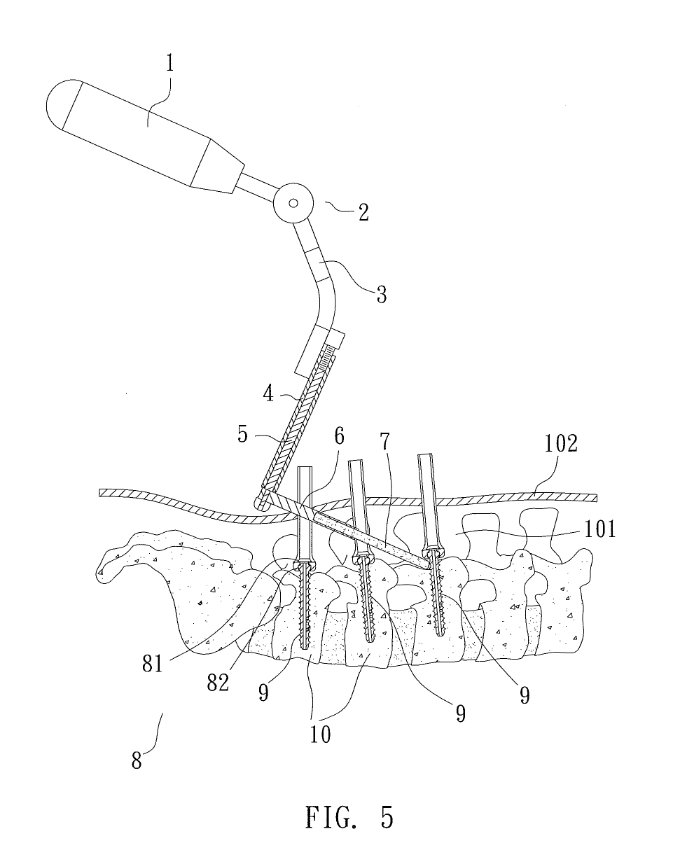

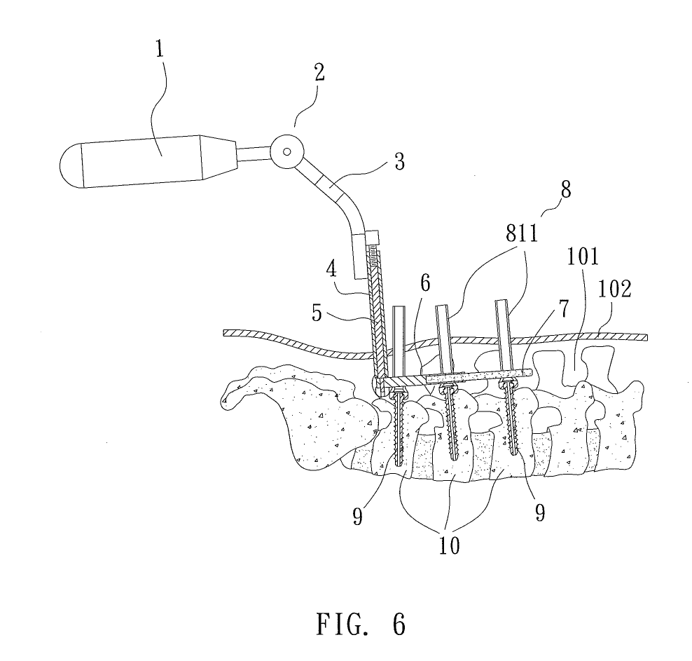

With reference to FIGS. 5 and 6, when the handle 1 is rotated in an anti-clock direction (the joint module 2 adjusts the desired angle between the handle 1 and the curved bar 3 so that the handle 1 does not interfere with a patient's body), such that the fixed rod clamping structure retained by the fitting post 4 and the positioning column 5 rotates in the muscle tissue 101 from a perpendicular state to a horizontal state (during which the skin 102 expends slightly), and then the fixed rod clamping structure is retained between two side walls 811 of the body 8 of the at least one bone screw so that the body 8 of the at least one bone screw is controlled by the fixed rod clamping structure, hence the at least one spine 10, which screws with the at least one screw section 9 on the front end of the body 8 of the at least one bone screw, is actuated to generate a rigid (i.e., at least one joint section between the rigid sleeve 6) and flexible (i.e., at least one joint section between the flexible shaft 7) engagement. Thereby, only a little wound is cut in the surgery to lower infection risk and to recover quickly.

While we have shown and described various embodiments in accordance with the present invention, it is clear to those skilled in the art that further embodiments may be made without departing from the scope of the present invention.

1. A fixed rod clamping structure for a minimally invasive surgery comprises:
a handle;
a joint module including a first sprocket and a second sprocket engaging with the first sprocket, with the first sprocket connecting with the handle;
a curved bar coupling with the second sprocket of the joint module;
a fitting post connecting with the curved bar and having an orifice and a through hole, wherein the orifice is perpendicular to and communicates with the through hole and has inner threads formed on one end thereof adjacent to the curved bar; and
a positioning column including a rotatable head, a pin, and outer threads formed on one end thereof adjacent to the rotatable head, wherein the outer threads are screwed with the inner threads of the orifice.

2. The fixed rod clamping structure for the minimally invasive surgery as claimed in claim 1, wherein the handle is axially connected with the curved bar by way of the first sprocket and the second sprocket of the joint module.

3. The fixed rod clamping structure for the minimally invasive surgery as claimed in claim 1, wherein the rotatable head of the positioning column includes a hexagonal aperture.
